(19) United States
(12) Patent Application Publication
(10) Pub. No.: US 2015/0314110 A1
(43) Pub. Date: Nov. 5, 2015

(54) BALLOON VISUALIZATION FOR TRAVERSING A VESSEL

(71) Applicant: Hansen Medical, Inc., Mountain View, CA (US)

(72) Inventor: June Park, San Jose, CA (US)

(73) Assignee: Hansen Medical, Inc., Mountain View, CA (US)

(21) Appl. No.: 14/269,649

(22) Filed: May 5, 2014

(51) Int. Cl. A61M 25/10; A61B 1/05; A61B 1/07; A61B 1/06

(52) U.S. Cl. CPC A61M 25/1002; A61B 1/0676; A61B 1/05; A61B 1/07; A61M 2025/1004

(57) ABSTRACT

Systems and methods for controllably traversing a tubular vessel, e.g., of a patient's vasculature. In one example, a distal end of a catheter is positioned and/or repositioned utilizing direct visualization out the distal end of the catheter, as facilitated by an imaging element disposed within the distal tip of the catheter. An inflatable balloon may comprise a portion of the distal tip of the catheter for structural and/or visualization media purposes. The balloon may define one or more channels configured to facilitate fluid flow between proximal and distal ends of the balloon after the balloon is inflated within the tubular vessel.

### BACKGROUND

One of the challenges in sending a medical device or portion thereof across an internal body tissue wall is ensuring that the device is not advanced too far past the tissue wall, which can damage adjacent tissue structures. The use of minimally invasive surgical techniques, such as those employing catheters or other elongate surgical probes, complicate this challenge by taking certain aspects of a given medical procedure beyond the normal field of view of the surgeon. For example, conventional minimally invasive techniques for placing a trocar or needle across the atrial septum of a heart involves pushing a transseptal needle, such as those sold by Medtronic/AVE under the tradename “Brockenbrough™”, out of a introducer sheath and across the atrial septum, with guidance provided by a conventional imaging modality, such as fluoroscopy.

While conventional techniques, such as “over-the-guidewire” techniques, enable approximate positioning of a transseptal needle adjacent a targeted location upon the atrial septum, there is still no assurance that the needle is correctly positioned before advancement through the tissue wall. Further, it is difficult ascertaining whether the tip of the transseptal device been advanced across the tissue wall and into an adjacent cavity, and whether the cavity is, in fact, the targeted cavity.

Known techniques for traversing a tissue walls include the use of inflatable balloon structures to permit visualization of the tissue wall and adjacent areas, e.g., as described in U.S. patent application Ser. No. 13/452,029. However, such balloon structures are not ideal for use in other, more constricted areas, e.g., within a vessel of a patient vasculature, since the balloon necessarily blocks flow through the vessel upon inflation. Accordingly, there is a need for an improved system and method for traversing a vessel within a patient vasculature that permits visualization while simultaneously permitting flow through the vessel.

### BRIEF DESCRIPTION OF THE DRAWINGS

The invention is illustrated by way of example and is not limited to the embodiments in the figures of the accompanying drawings, in which like references indicate similar elements. Further, features shown in the drawings are not intended to be drawn to scale, nor are they intended to be shown in precise positional relationship.

FIG. 1A depicts a side view of a catheter structure in accordance with one embodiment of the invention.

FIG. 1B depicts a cross-sectional view of the structure of FIG. 1A.

FIG. 1C depicts a cross-sectional view of a catheter structure in accordance with another embodiment of the invention.

FIG. 1D depicts a side view of a catheter structure in accordance with yet another embodiment of the invention.

FIG. 1E depicts a side view of a catheter structure in accordance with still another embodiment of the invention.

FIG. 1F depicts a side view of a catheter structure in accordance with yet another embodiment of the invention.

FIG. 2 depicts a side view of a catheter structure in accordance still another embodiment of the invention.

FIGS. 3A-3F are side views depicting various stages of yet another catheter structure embodiment of the invention, including a traversing member traversing a tissue wall in a body.

FIGS. 3G-3I are side views of a variation of the embodiment of FIGS. 3A-F, in which sleeve remains positioned after withdrawal of the tissue traversing member to function as an access lumen across the tissue wall.

FIG. 4 is a side view depicting use of a catheter structure embodiment similar to that depicted in FIG. 2.

FIGS. 5A-5N depict various structures for confirming a position of a traversing member relative to a tissue wall in accordance with various embodiments of the invention.

FIGS. 6A and 6B are flow charts illustrating two exemplary procedures for traversing a tissue wall in accordance with embodiments of the invention.

FIG. 7 is a schematic illustration of an exemplary catheter structure including a balloon defining one or more channels along an exterior surface of the balloon;

FIGS. 8A and 8B are a sectional view and a perspective view, respectively, of an exemplary balloon structure for the catheter of FIG. 7;

FIGS. 9A and 9B are a sectional view and a perspective view, respectively, of another exemplary balloon structure for the catheter of FIG. 7; and

FIG. 10 is a flow chart illustrating an exemplary method for traversing a tubular vessel.

### DETAILED DESCRIPTION

In the following detailed description of various exemplary illustrations, reference is made to the accompanying drawings in which like references indicate similar elements. The illustrative examples described herein are disclosed in sufficient detail to enable those skilled in the art to practice the invention. The following detailed description is therefore not to be taken in a limiting sense, and the scope of the invention is to be defined and limited only by the appended claims.

As noted above, various U.S. patent application Ser. No. 13/452,029, filed Apr. 20, 2012, which is a continuation of U.S. patent application Ser. No. 10/949,032, filed Sep. 24, 2004 and now U.S. Pat. No. 8,172,747, which claims priority to Provisional Application Ser. No. 60/506,293, filed Sep. 25, 2003, and the contents of each of these applications are fully incorporated herein by reference in their entireties

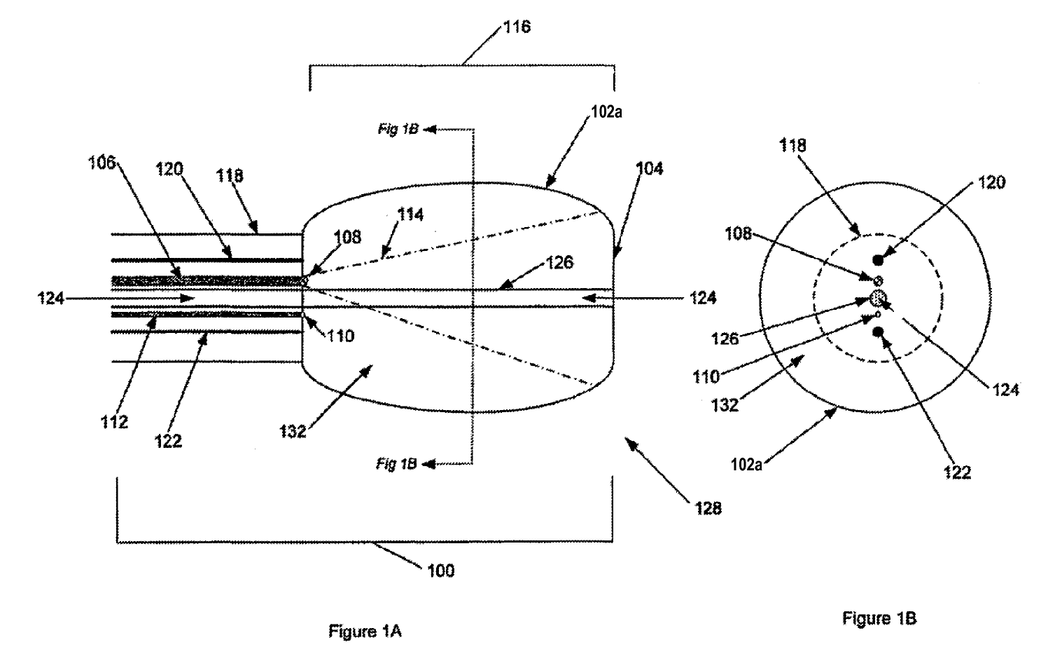

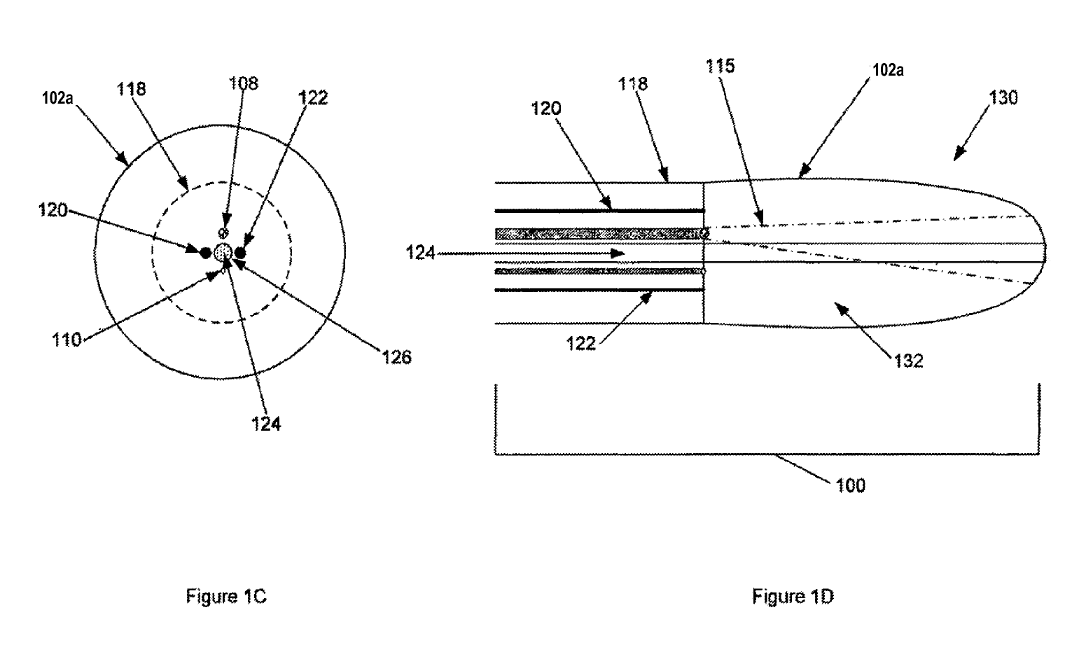

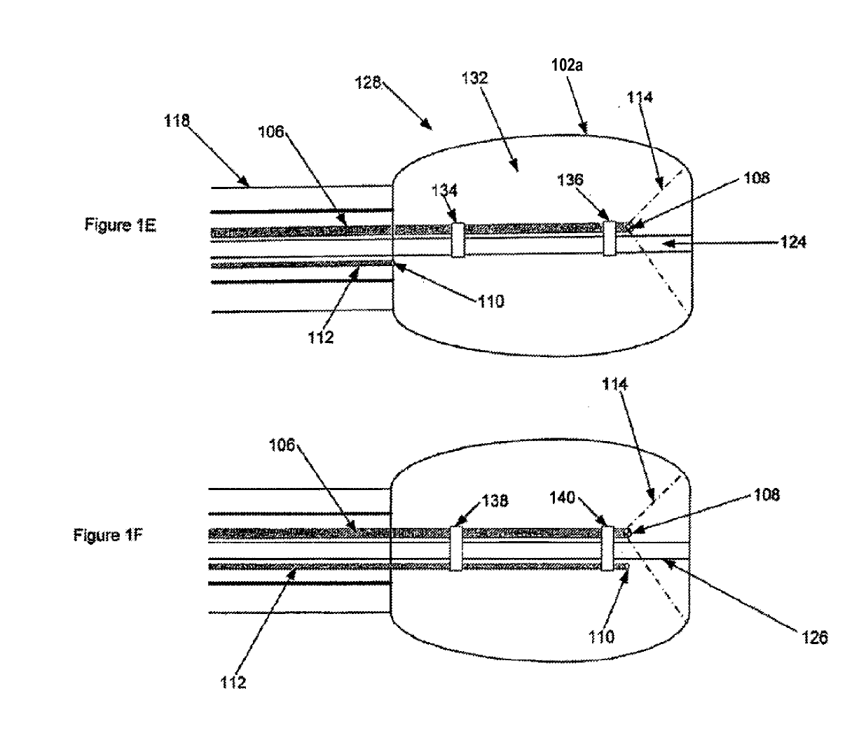

Referring to FIG. 1A, a side view of a distal portion (100) of a catheter in accordance with the present invention is depicted. The same structure is depicted in cross-sectional view in FIG. 1B. In the depicted embodiment, a balloon (102a) is disposed at the end of an elongate tubular member (118) which defines various lumens (120, 122, 124) and confines various structures (106, 112) associated with balloon-based optical visualization. A working lumen (124), defined in part by the elongate tubular member (118) and in part by an associated tubular member (126), provides continuous access down the longitudinal axis of the distal portion (100) of the catheter. The tubular member (126) extends from the elongate tubular member (118) to the distal end surface (104) of the balloon, which in the depicted embodiment is substantially flat with the balloon (102a) in an inflated or expanded configuration (128). The balloon (102a), preferably comprising a translucent polymeric material such as nylon and filled with saline (132) or some other substantially translucent and biologically inert low-viscosity fluid by inflation through one or more balloon sizing lumens (120, 122), provides a medium through which an imaging element (108) may capture images of the tubular member (126) and adjacently positioned objects, such as tissue structures, which fall within the field of view (114) of the imaging element. In another embodiment, the balloon (102a) may be inflated with carbon dioxide or another relatively biologically inert gas.

The tubular member (126) also preferably comprises a substantially translucent polymeric material, such as polymethylmethacrylate (“PMMA”) or polyimide, paired appropriately with the imaging modality, to enable visualization into and across the tubular member (126) with the imaging element (108). The tubular member may comprise a separate tube component coupled to the distal end of the elongate tubular member (118) utilizing conventional techniques such as polymeric adhesive or stainless steel clips, or may comprise an extension of the material comprising the elongate tubular member (118).

The imaging element (108) may comprise a distal end of an optical fiber, in which case the depicted image transmission line (106) comprises an optical fiber, or it may comprise another image capturing element, such as a charge-coupled-device (CCD) or infrared imaging chip, in which case the image transmission line (106) may comprise an electronic data transmission wire. A lighting element (110) is paired with the imaging element to provide illumination or radiation appropriate for capturing images in the given tissue cavity. In the case of an optical fiber distal end as an imaging element (108), the lighting element (110) preferably comprises an emitter of light, such as a small light bulb, light emitting diode, or end of another optical fiber distal end in communication with an emitter or light. The light energy transmission line (112) may comprise optical fiber, electronic lead wire, or the like to transmit the appropriate lighting energy to the lighting element (110). In another embodiment, the lighting element (110) comprises an emitter of infrared-spectrum radiation and the imaging element (108) comprises an infrared-detecting imaging element to enable infrared-spectrum visualization within the geometrically prescribed field of view (114). Suitable infrared emitters and detectors are well known in the art and available from suppliers such as CardioOptics of Boulder, Colo.

The imaging element (108) may comprise a lens, filter, mirror, or other structure configured to control the field of view (114) or focal length of the associated imaging element (108). Further, a lens, filter, mirror, or other structure may be positioned distally from the imaging element (108) within the balloon portion (116) of the catheter distal end (100) for similar purposes. The utilization of a imaging element (108) located at the distal end of a medical instrument, such as a balloon catheter, for purposes of visualizing objects from the point of interest is referred to herein as “direct visualization”. In other words, “direct visualization” is used in reference to placing an imaging “eye” distally to the location of tissue treatment interest.

Referring to FIG. 1B, a cross-sectional view of the structures of FIG. 1A depicts the balloon sizing lumens (120, 122), working lumen (124), imaging element (108), and lighting element (110) in a substantially aligned configuration which is more resistive to cantilever bending of the catheter distal end (100) along the direction of the aligned configuration than in a direction 90-degrees rotated from such alignment. In another embodiment, as shown in FIG. 1C, such componentry is cross-sectionally arranged as tightly as possible about the central axis of the elongate tubular member (118) to facilitate easier and more uniform cantilever bending. Such components may be arranged within the elongate tubular member (118) to facilitate overall mechanical performance goals given the mechanics of the components themselves. For example, in an embodiment where high cantilever-bending flexibility in all directions is desired, and wherein the image transmission line (106) and light energy transmission line (112) comprise relatively stiff optical fiber, it is advantageous to position these two structures (106, 112) close to the central axis of the elongate tubular member (118).

The elongate tubular member (118) preferably comprises a conventional polymeric material, such as that sold under the trade name “Pebax™” by Atofina Corporation, which is suitable for use inside of animals and has desirable mechanical and manufacturing properties. In the case of optical fiber, glass fibers, such as those conventionally utilized in endoscopes, may be utilized, or more flexible polymeric optical fibers, such as those available from Nanoptics Corporation of Gainesville, Fla., may be utilized.

FIG. 1D depicts a structure similar to that of FIG. 1A, with the exception that the balloon (102a) is in a deflated or contracted configuration (130), as the result of a removal of saline (132) from the balloon (102a) via the balloon sizing lumens (120, 122). In one embodiment, one of the balloon sizing lumens (120) is reserved only for inflation, while the other (122) is reserved for deflation. More or less than two balloon sizing lumens may be suitable, depending upon the diameter of such structures and desired rate of inflation and deflation, as would be apparent to one skilled in the art. As shown in FIG. 1D, the contracted (130) balloon preferably has an outer diameter roughly the same size as the associated elongate tubular member (118) for atraumatic, smooth endolumenal delivery while also facilitating a narrowed forward-looking field of view (115) with the imaging element (108) during delivery.

Referring to FIG. 1E, another embodiment is depicted wherein the imaging element (108) is positioned forward within the balloon (102a) to gain better access to adjacent objects of interest adjacent the distal end of the balloon (102a) and within the field of view (114) of the imaging element (108) and broadcast range of the lighting element (110). With such a configuration the image transmission line (106) extends beyond the distal end of the elongate tubular member (118) and into the balloon (102a), as depicted in FIG. 1E. The portion of the image transmission line (106) within the balloon (102a) may be mechanically stabilized with small polymer or metallic clips (134, 136), as shown.

Referring to FIG. 1F, another embodiment is depicted wherein the lighting element (110) is positioned forward into the balloon (102a) with the imaging element (108) to minimize shadowing effects. Mechanical stabilizers (138, 140) similar to those described in reference to FIG. 1E may be utilized to maintain the relative positioning of the balloon (102), tubular member (126), imaging element (108), and lighting element (110).

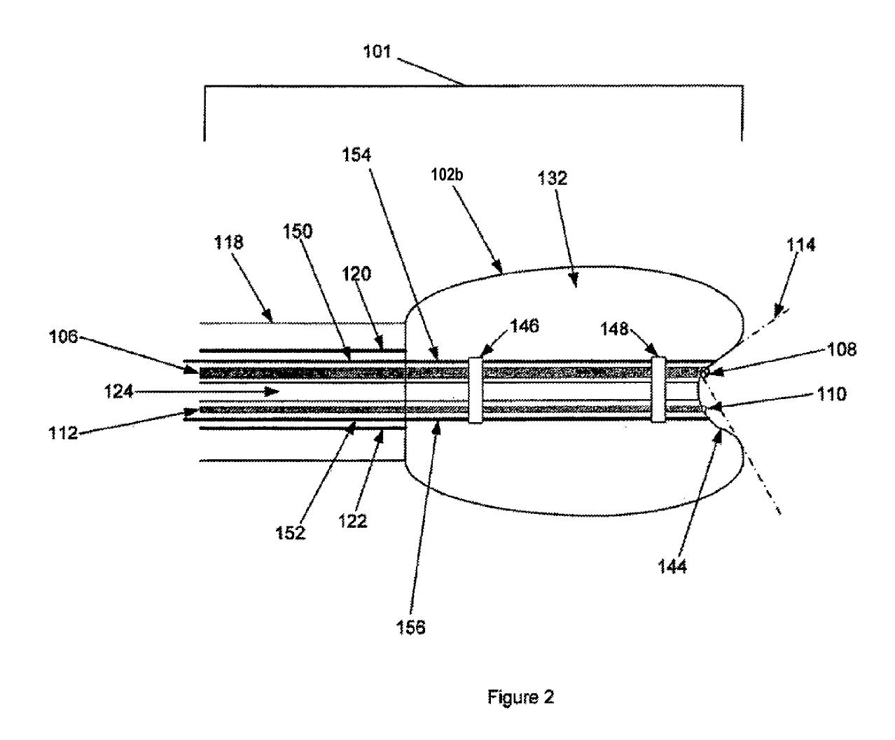

Referring to FIG. 2, another embodiment is depicted wherein a balloon (102b) comprises a concave surface (144) distally upon inflation, and wherein the imaging element (108) and lighting element (110) are positioned for substantially immediate visualization of adjacently positioned objects. With such a configuration, it may be necessary or preferable to flush and fill a volume defined by the concave surface (114) and an immediately interfaced object with a translucent, high-viscosity, and biologically inert fluid such as saline, or biologically inert gas, utilizing additional fluid delivery lumens such as those depicted (150, 152) in FIG. 2. Such lumens (150, 152) are extended through the balloon (102b) to the concave surface (144) by small tubular members (154, 156), which may be stabilized along with the other associated structures within the balloon (102b) utilizing mechanical stabilizers (146, 148) similar to those described in reference to FIG. 1E, as shown in FIG. 2.

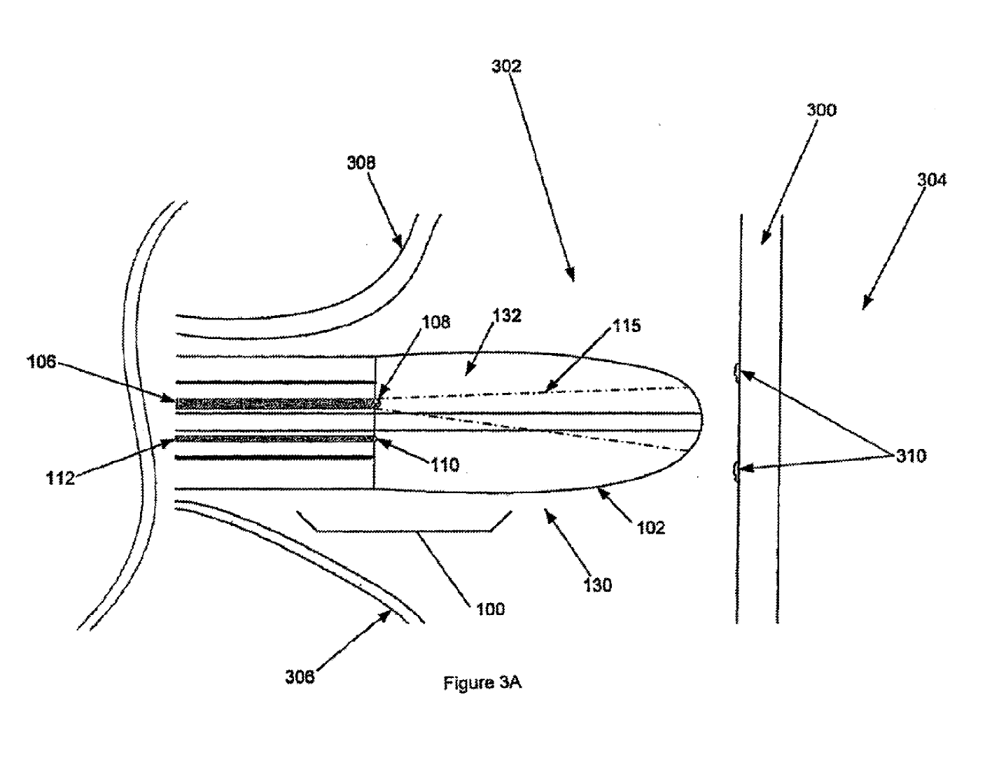

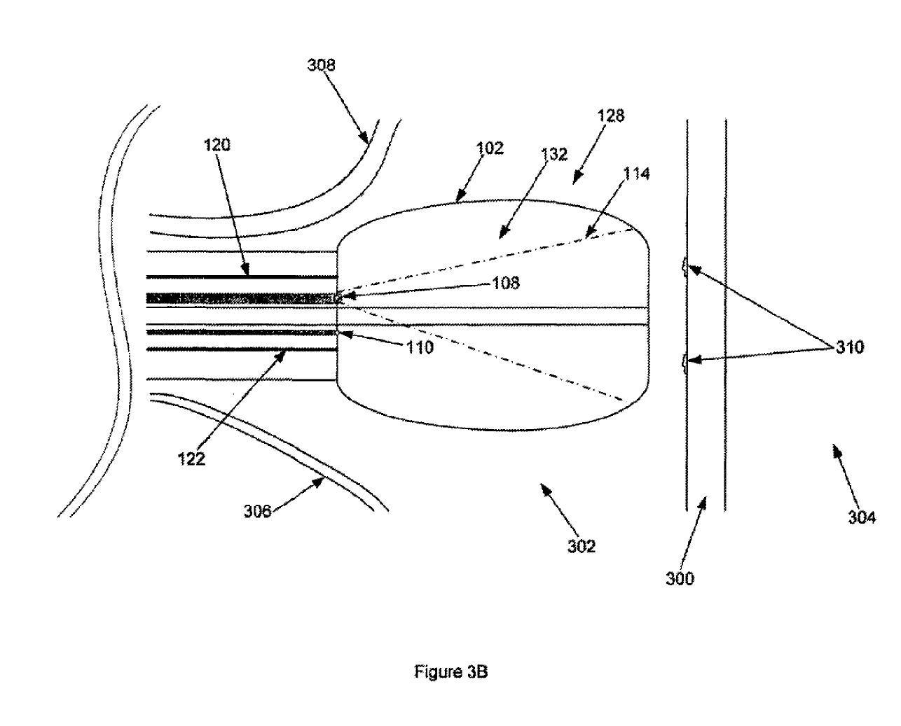

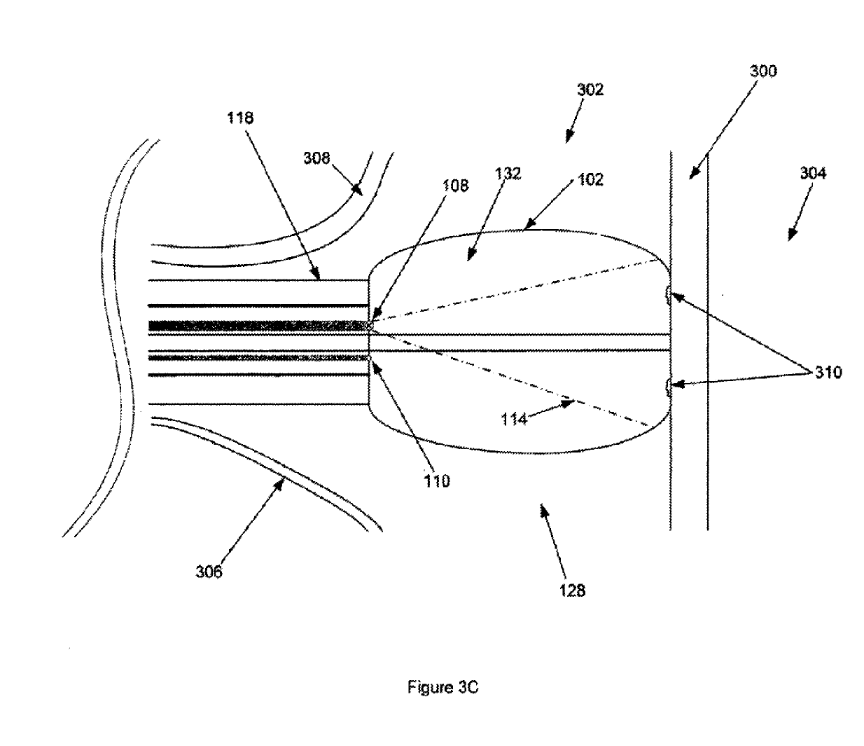

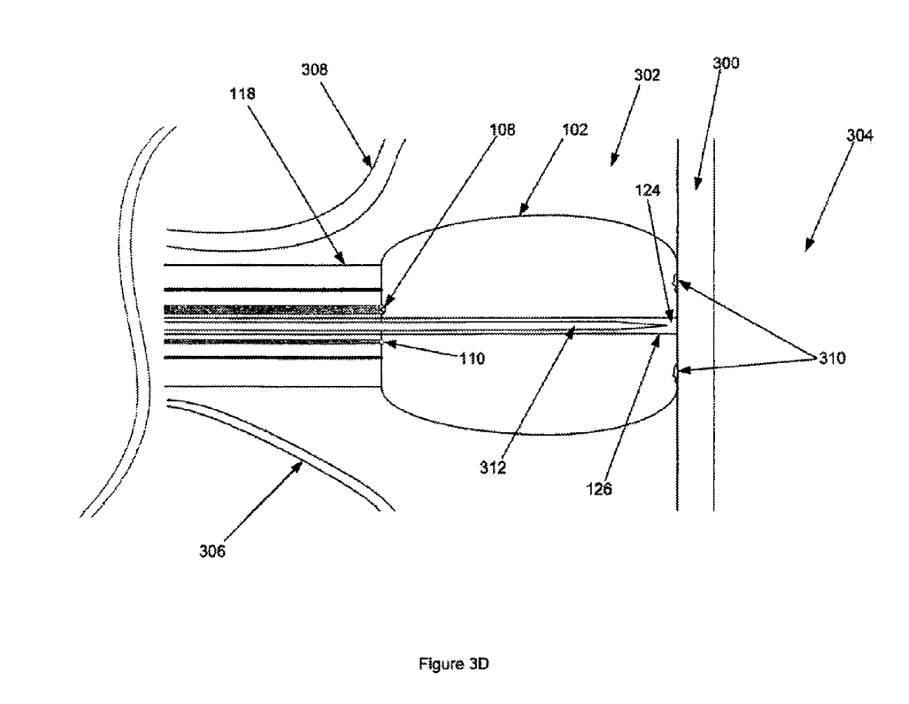

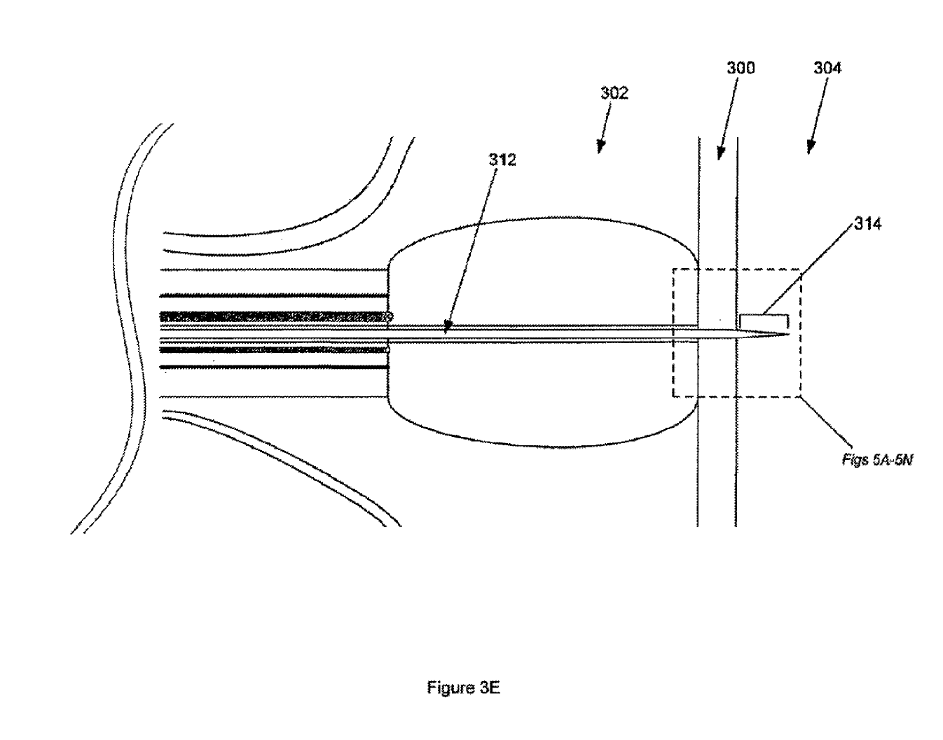

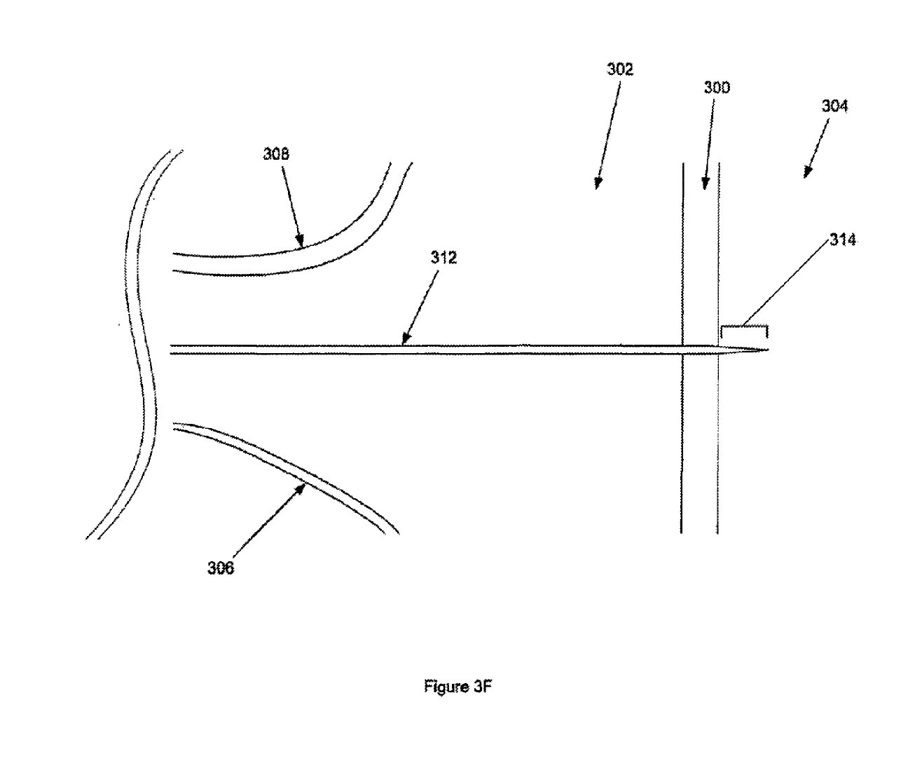

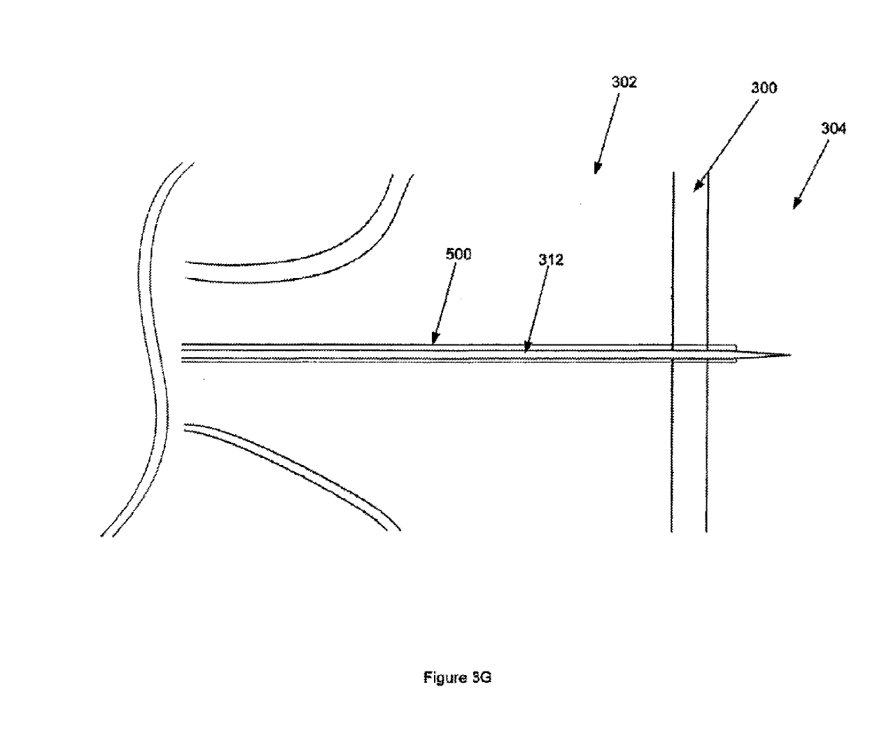

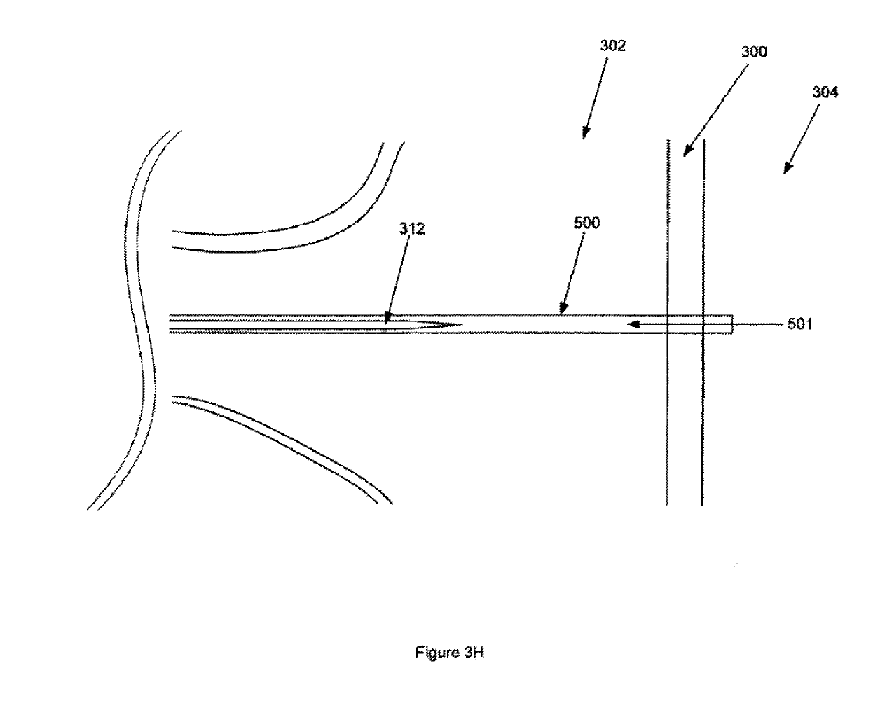

Referring to FIGS. 3A-3F, one embodiment of a method for using structures such as those described in reference to FIGS. 1A-1F is depicted. Utilizing conventional techniques, the distal end of the catheter (100) is positioned in the vicinity of a targeted tissue structure or tissue wall (300). As shown in FIG. 3A, the distal end (100) may be advanced through a relatively tight geometry between two tissue structures (306, 308) and into a first enlarged cavity (302), which may be opposite the targeted tissue wall (300) from a second enlarged cavity (304). A targeted region of the tissue wall (300) may be identifiable by terrain or substructures (310) comprising the surface of the targeted tissue wall (300).

As the contracted (130) balloon (102a or 102b; collectively referred to as 102) approaches the substructures (310), a narrowed field of view (115) captured by the imaging element (108) as facilitated by the lighting element (110) may be utilized for navigating the balloon (102) into position adjacent the tissue wall (300). In a substantially nontranslucent media such as blood within the first cavity (302), visualization of the substructures or tissue wall may not be useful until the distal end of the balloon is very close to the tissue wall (300), whereas in a more translucent media, such as saline or carbon dioxide, targeted tissues and substructures may become visible as soon as they are within a direct field of view, depending upon the focal characteristics of the imaging element (108), as would be apparent to one skilled in the art. Further, the translucent media within the balloon (102) may comprise a contrast agent to facilitate imaging. For example, in the case of a conventional fluoroscopic imaging modality, the translucent media preferably comprises a conventional contrast agent such as iodine.

Upon entry into a relatively large cavity (302), the balloon may be inflated to provide a broadened field of view and illumination, as shown in FIG. 3B. Positioning of the balloon (102) relative to the first enlarged cavity (302) may be confirmed or monitored using conventional techniques, such as ultrasound and fluoroscopy.

Referring to FIG. 3C, the expanded balloon is advanced into contact with the targeted tissue wall (300), where even with a nontranslucent cavity (302) media such as blood, the tissue wall (300) and substructures thereof (310) may be visualized through the balloon (102) with the imaging element (108) and lighting element (110). With such visualization, it may be preferable to fine-tune the position of the balloon relative to the tissue wall (300).

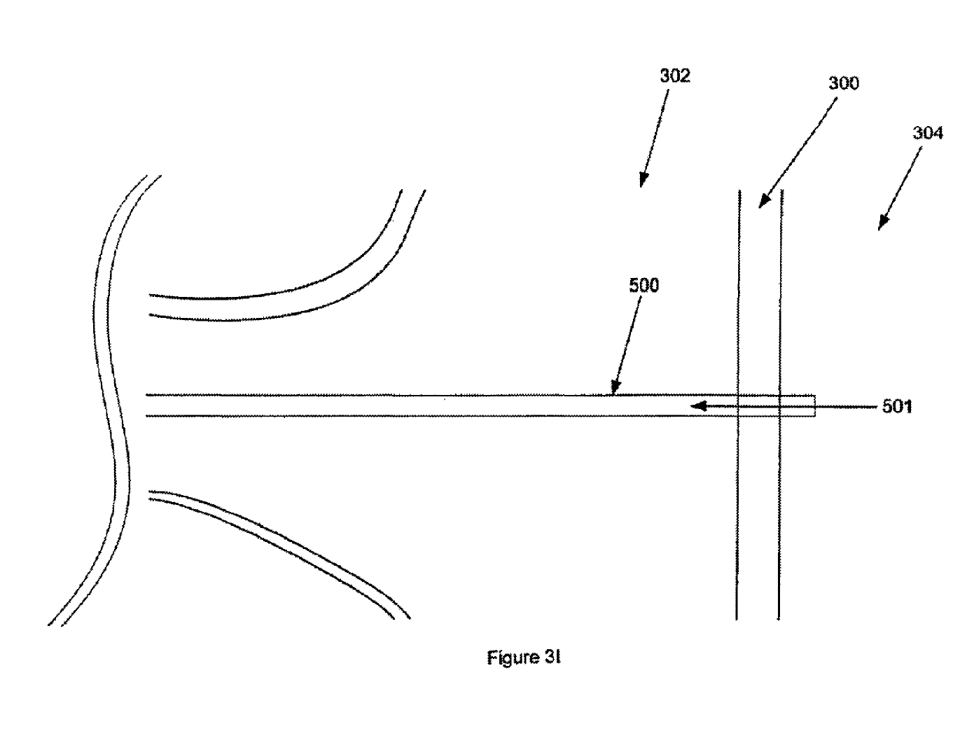

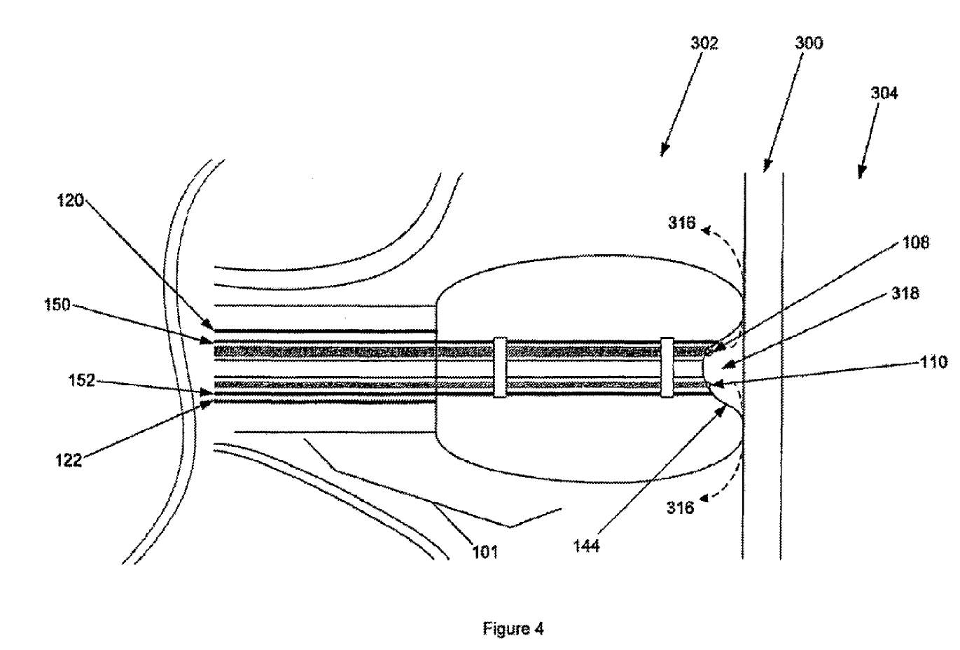

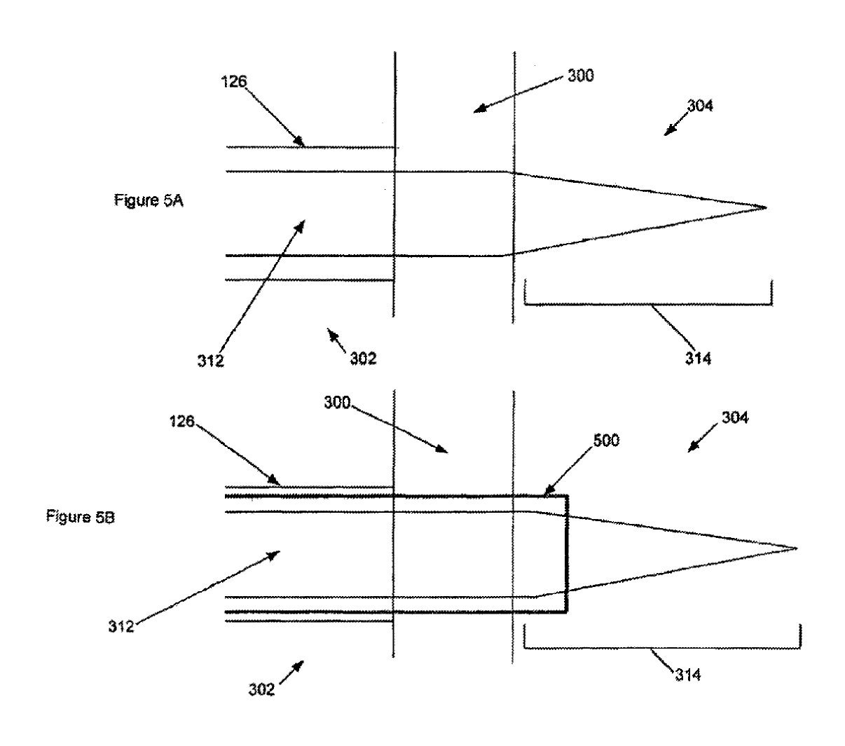

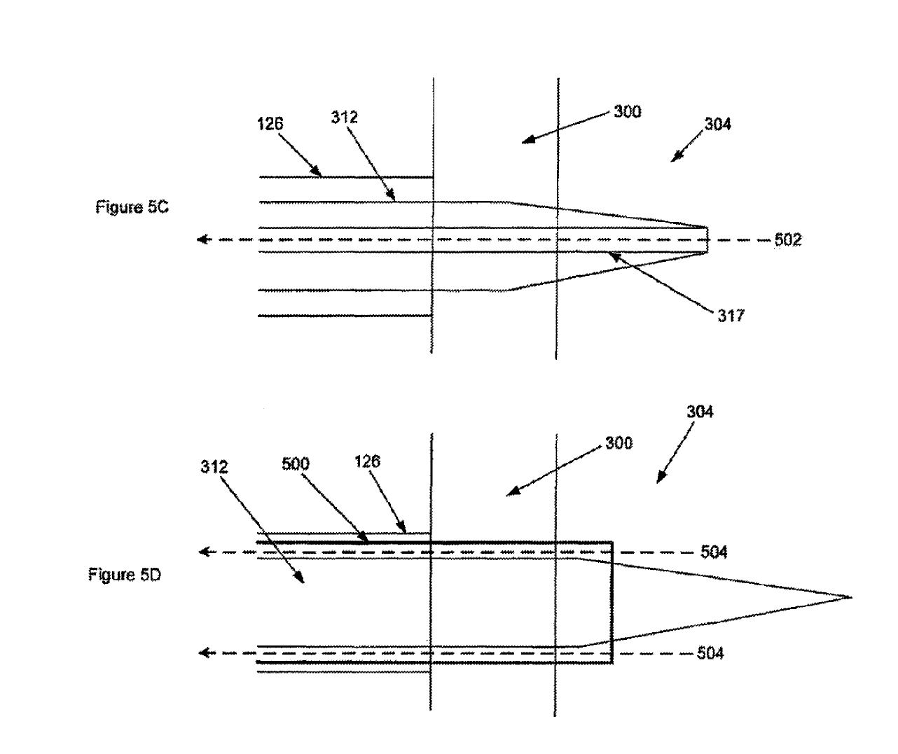

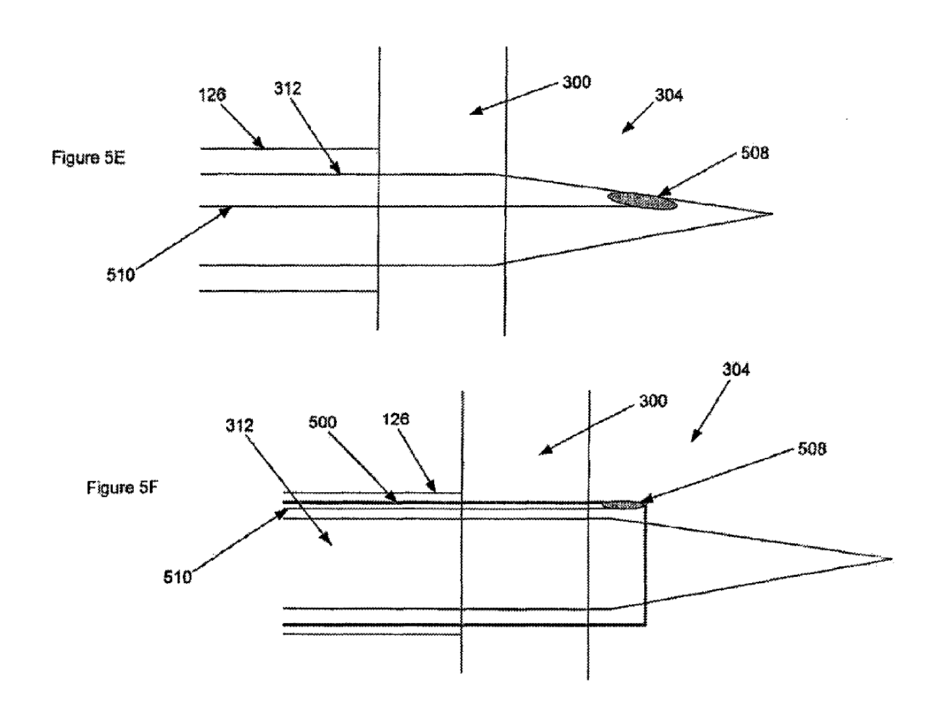

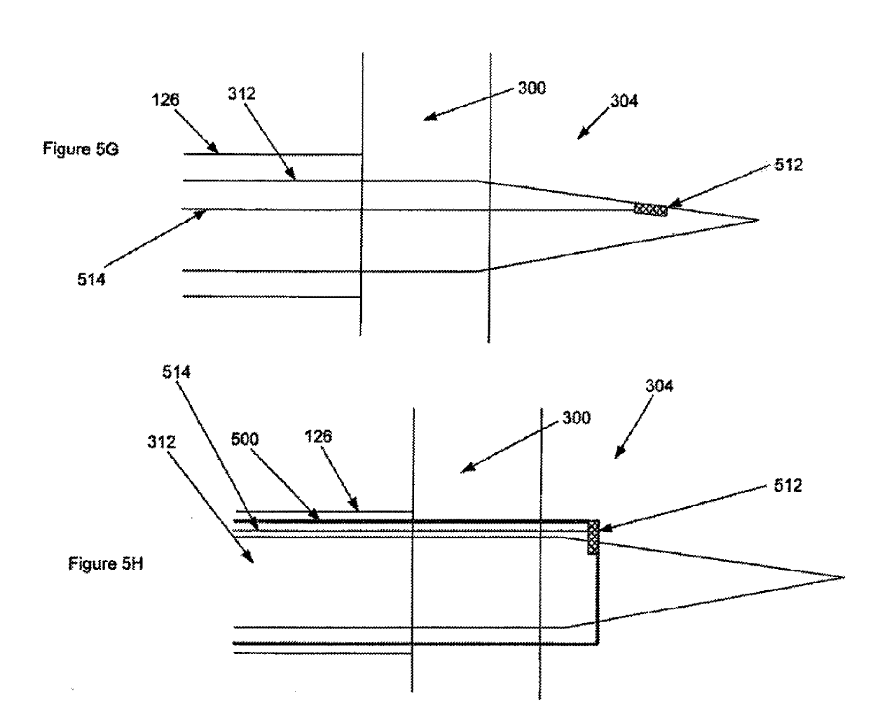

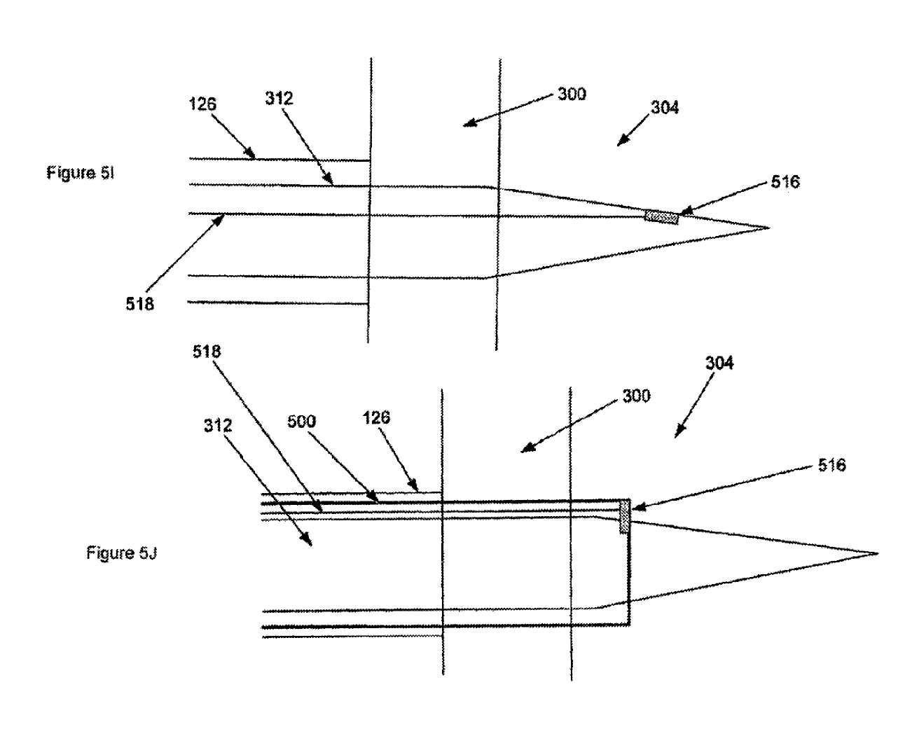

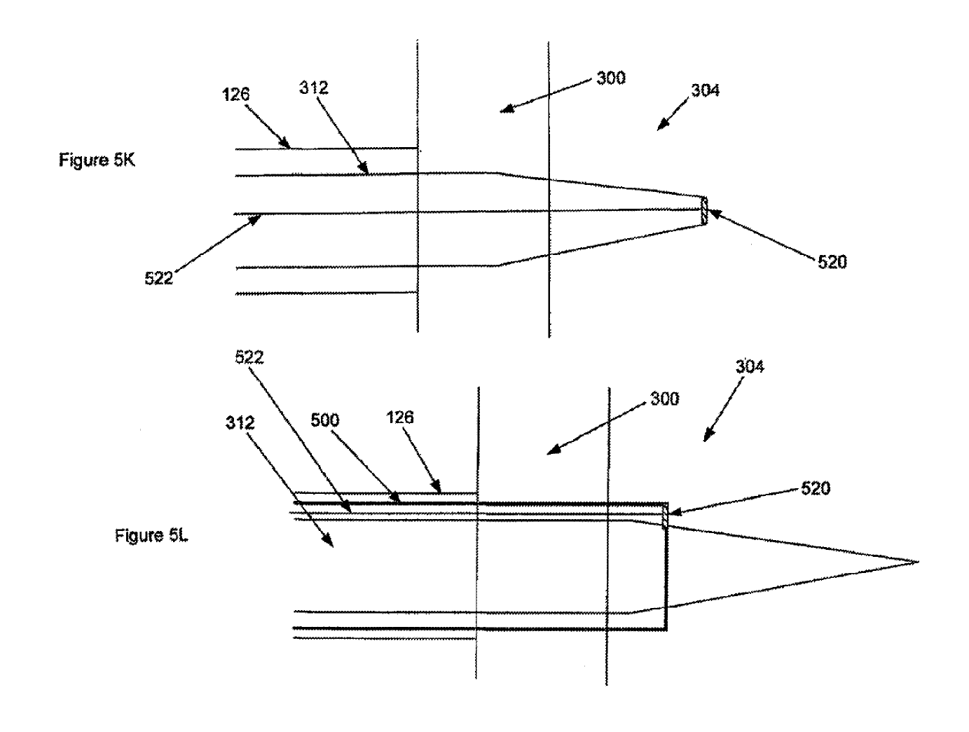

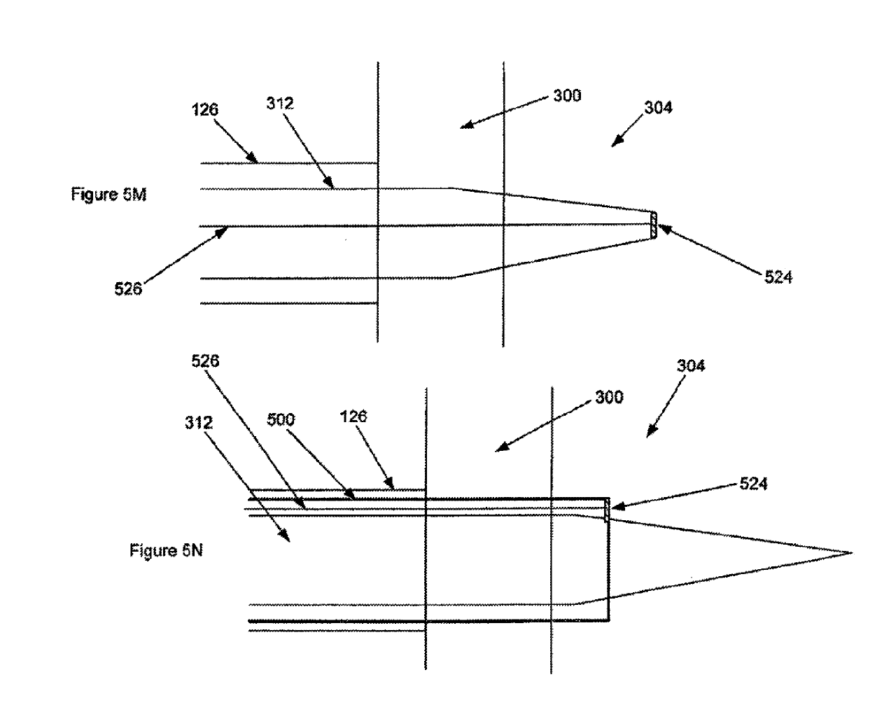

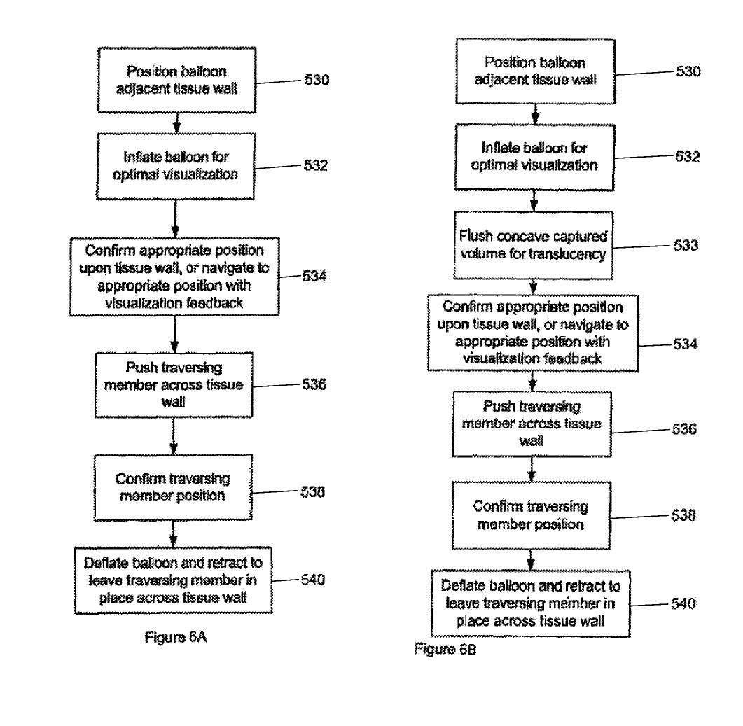

Referring to FIG. 3D, a tissue traversing member, such as a trocar or Brockenbrough™ needle, is advanced through the working lumen (124) of the elongate tubular member (118) and tubular member (126) to a position adjacent the targeted tissue wall (300). As described in reference to FIGS. 1A and 1B, the tubular member (126) preferably comprises a material through which the imaging element (108) can “see” to provide the user with feedback regarding the relative positioning of the traversing member (312), balloon (102), and tissue wall (300). After confirmation of preferred alignment of these structures, the traversing member (312) is advanced into and across the tissue wall (300), as depicted in FIG. 3E. Visualization of gradient markers (not shown) on the traversing member (312), along with images of the pertinent structures from other modalities, such as fluoroscopy and/or ultrasound, facilitate precise positioning of a portion of the traversing member (312) across and beyond (314) the subject tissue wall (300), into the second cavity (304). Further details of the traversal positioning and confirmation are described in reference to FIGS. 5A-5N. While the illustrative description in reference FIGS. 3A-3E incorporates a balloon catheter structure similar to that of FIG. 1A, such description is applicable to embodiments such as those depicted in FIGS. 1E and 1F and FIG. 2, with the exception that the structures similar to FIG. 2 may involve additional steps, as further described in reference to FIG. 4 and FIG. 6B.

Referring to FIG. 3F, subsequent to traversal and confirmation of desired positioning of the traversing member (312), the distal end of the catheter may be withdrawn, leaving behind the traversing member (312). As depicted in FIG. 3G, in another embodiment, the traversing member (312) is advanced into place accompanied with a preferably tubular sleeve (500), which may remain in place along with the traversing member (312) following withdrawal of the catheter. Referring to FIGS. 3H and 3I, the traversing member (312) may subsequently be withdrawn, leaving behind only the sleeve (500), which provides an access lumen (501) over to the second cavity (304), the access lumen being usable as a working lumen for tools, injections, and the like which may be used to examine and treat the second cavity (304), tissue wall (300), or other adjacent tissues.

Referring to FIG. 4, a depiction of a catheter distal end (101) similar to that depicted in FIG. 2 is illustrated in a position analogous to the positioning of structures of FIG. 3C to illustrate the notion that a flushing of substantially translucent fluid (316) may be utilized to facilitate viewing by the imaging element (108) and lighting element (110) through a volume (318) captured between the concave surface (144) of the depicted embodiment and a tissue wall (300).

Referring to FIG. 5A, a close-up side view depicting an embodiment of a traversing member (312) positioned across a tissue wall (300) is depicted. As shown in FIG. 5A, the traversing member (312) is guided to the tissue wall (300) by the tubular member (126). The distal tip (314) of the traversing member is positioned in the cavity (304) opposite the cavity (302) in which the tubular member (126) is positioned. Referring to FIG. 5B, a close-up side view depicting an embodiment of a traversing member (312) with a sleeve (500) is illustrated, with the sleeve (500) and traversing member (312) both advanced into a position across the tissue wall with distal protrusion (314) into the cavity (304) opposite the cavity in which the tubular member (126) is positioned.

FIGS. 5C-5D, 5E-5F, 5G-5H, 5I-5J, 5K-5L, and 5M-5N depict pairings of embodiments analogous to the sleeveless and sleeved embodiments depicted in FIGS. 5A and 5B. Each of these pairings features a different embodiment for confirming the position of a traversing member (312) across a subject tissue wall (300) by sensing or monitoring a difference known to be associated with a desired second cavity (304) position or position within the targeted tissue wall (300). For example, localized pressure within a first cavity (302) may be substantially different than both the localized pressure within the tissue wall (300) and within a second cavity (304).

Likewise, for flow rate, oxygen saturation, etcetera, as described in reference to FIGS. 5C-5N. Each of the different monitoring variables is described separately in FIGS. 5C-5N, but, as would be apparent to one skilled in the art, the monitoring structures and modalities may be combined for increased position determination capability. For example, in one embodiment it is desirable to sense both pressure changes and echo timing for redundancy in determining whether the distal tip (314) of a traversing member (312) and/or sleeve (500) is within a first cavity (302), tissue wall (300), second cavity (304), or perhaps an undesirable location in a third cavity, such as a major blood vessel with a substantially high flow rate as detected by Doppler and distinguished from a targeted destination cavity.

Referring to FIG. 5C, the location of the traversing member (312) may be determined by sampling fluid along a pathway (502) through a lumen (317) formed in the traversing member (312), and transporting sampled fluid proximally to a position outside of the body for conventional testing, such as rapid chemical testing, pulse oximetry, and the like. A traversing member (312) defining such a lumen (317) could be formed using conventional technologies, and purchased from suppliers of high-precision machined trocars and similar structures such as Disposable Instrument Company, Inc. of Shawnee Mission, Kans. FIG. 5D depicts a sleeved embodiment wherein fluid is sampled along a pathway (504) between the traversing member (312) and the sleeve (500). Referring back to FIG. 3I, the lumen (501) of an empty sleeve (500) may serve a similar purpose.

Referring to FIG. 5E, a traversing member (312) having a distally disposed pressure sensor (508) with a sensor lead (510) may be utilized to monitor pressure changes at the distal portion of the traversing member (312). Suitable small pressure sensors (508) are known in the art and available from suppliers such as Motorola Sensor Products of Phoenix, Ariz., and IC Sensors, a division of Measurement Specialties, of Milpitas, Calif. FIG. 5F depicts an embodiment wherein a pressure sensor (508) is coupled to a sleeve (500).

Referring to FIG. 5G, a color shade sensor (512) is coupled to the distal portion of a traversing member (312) to facilitate monitoring of the color or graytone of local objects such as arterial versus venous blood. The color shade sensor (512) preferably comprises a CCD or CMOS-based image sensor, such as those available from suppliers such as Eastman Kodak Image Sensor Solutions, which is configured in this embodiment to transmit data proximally through a sensor lead (514) as depicted. FIG. 5H depicts an embodiment wherein a color shade sensor (512) is coupled to a sleeve (500) as opposed to directly to the traversing member (312). As would be apparent to one skilled in the art, mirrors, lenses, filters, and the like may be utilized to enhance or modify the image sampling of such sensors.

Referring to FIG. 5I, an oxygen saturation sensor (516) is coupled to the distal end of a traversing member (312) to facilitate monitoring of the partial pressure of oxygen at the oxygen saturation sensor (516) location utilizing a sensor lead (518). As shown in FIG. 5J, an oxygen saturation sensor (516) may also be positioned upon a sleeve (500). Small oxygen saturation sensors, generally comprising a small radiation transmitter, such as a laser diode, and a small radiation receiver, are available from suppliers such as Nellcor Puritan Bennett of Pleasanton, Calif.

Referring to FIG. 5K, an embodiment of a traversing member is depicted with a flow sensor (520) disposed at the distal tip, in a configuration selected to access flows straight ahead of the advancing traversing member (312). FIG. 5L depicts a similar embodiment with a flow sensor coupled to a sleeve (500). In each embodiment, flow rate data is transmitted proximally, preferably via a sensor lead (522). Small flow rate sensors, based upon Doppler ultrasound or laser diode technology are well known in the art and available from suppliers such as Transonic Systems, Inc. of Ithaca, N.Y.

Referring to FIG. 5M, an embodiment of a traversing member is depicted with an echo time sensor (524) disposed at the distal tip, in a configuration selected to access flows straight ahead of the advancing traversing member (312). FIG. 5N depicts a similar embodiment with an echo time sensor (524) coupled to a sleeve (500). In each embodiment, echo time data is transmitted proximally, preferably via a sensor lead (526). An echo time sensor (524), generally comprising a radiation emitter and detector configured to detect the proximity of objects in a manner similar to that of sonar technology, may be interfaced with a computer-generated sound signal which changes frequency in accordance with changes in echo time. With such a configuration, for example, the sound frequency when the echo time sensor (524) is positioned within a relatively high-density tissue wall (300) may vary significantly from the sound frequency associated with a relatively low density, open cavity (304), thereby facilitating detection of the distal end of a traversing member (312) as it is advanced across the tissue wall (300) and into the adjacent open cavity (304).

Referring to FIG. 6A, an embodiment of a tissue wall traversal process in accordance with the present invention is summarized in flowchart format. Referring to FIG. 6A, a balloon structure is positioned adjacent a tissue wall (530). Inflation of the balloon optimizes visualization by providing a relatively unobstructed field of view (532). Subsequent to confirming an appropriate position upon the tissue wall or navigating to an appropriate position utilizing visualization feedback (534), the traversing member is advanced across the tissue wall while the position is monitored for confirmation of appropriate positioning (538). With the traversing member appropriately positioned across the tissue wall, the balloon is deflated and retracted proximally to leave the transecting member in place across the tissue wall (540).

Referring to FIG. 6B, another embodiment of a tissue wall traversal process in accordance with the present invention is summarized in flowchart format. The embodiment of FIG. 6B differing from that of FIG. 6A in that the embodiment of FIG. 6B comprises an additional step of flushing a volume entrapped between a concave balloon surface to provide better translucency for image capture, in a process wherein a structure similar to that described in reference to FIGS. 2 and 4 is utilized.

A process similar to that of FIG. 6A or 6B may be utilized, for example, in a transseptal crossing procedure wherein safe access to the left atrium of the heart is desired. Referring back to FIG. 3A, in such an embodiment, tissue structures 306 and 308 may represent portions of the wall of a right atrium cavity (302), the tissue wall (300) may represent the atrial septum, and the second cavity (304) may represent the left atrium of the heart. In accordance with the aforementioned techniques and structures, the collapsed catheter distal end (100) may be advanced toward the atrial septum, guided into position by wall (300) terrain (310) such as the outline of the fossa ovalis.

Appropriate positioning of the working lumen (124) relative to the outlines of the fossa ovalis may be confirmed utilizing images from the imaging element (108) with a preferably fully expanded (128) balloon (102) urged against the atrial septum, subsequent to which a traversing member (312), such as a Brockenbrough™ needle, may be advanced into the atrial septal wall through the working lumen (124), as observed through the tubular member (126), and preferably also through redundant visualization modalities, such as ultrasound and/or fluoroscopy. Further, the traversing member (312) may be instrumented with a sensor, such as a pressure, flow rate, color shade, or other sensor, to confirm that the distal tip of the traversing member (312) is indeed where the operator thinks it is.

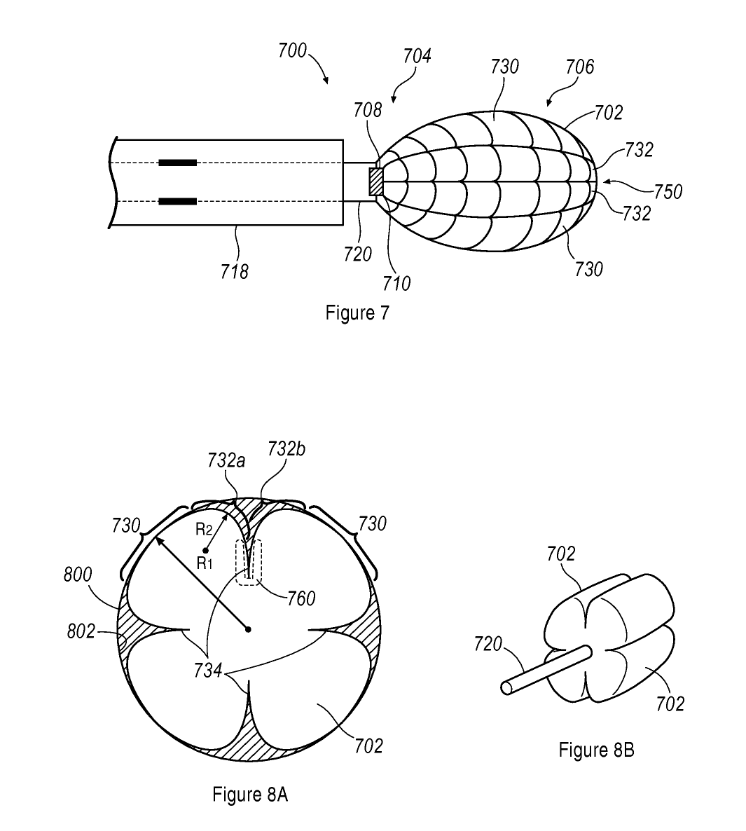

Turning now to FIGS. 7, 8A, and 8B, another exemplary illustration of a catheter assembly is shown. A distal portion (700) of the catheter is shown, and may include a selectively inflatable balloon (702) disposed at the end of an elongate tubular member (718). The tubular member (718) may be a guide catheter configured to be inserted and articulated within a vessel (not shown in FIG. 7) of a patient vasculature. The tubular member (718) may define various lumens similar to those described above in other exemplary illustrations, e.g., for providing access from a proximal end of the catheter to the distal end (700). A balloon catheter (720) may be received within the tubular member (718), and may be connected with the balloon (702). The balloon catheter (720) may be configured to provide continuous access to the balloon (702) along a longitudinal axis of the distal portion (700) of the catheter.

As with other exemplary balloons described above, the balloon (702) may comprise a translucent polymeric material such as nylon, and may be selectively filled with saline or some other substantially translucent and biologically inert low-viscosity fluid by inflation through one or more balloon sizing lumens (not shown in FIG. 7). More specifically, one or more balloon sizing lumens may be provided in a proximal end of the balloon (702), similar to the other exemplary balloons described herein. The balloon (702) may thereby provide a medium through which an imaging element (708) may capture images of the balloon (702) and adjacently positioned objects, such as interior walls of a vessel (800) (see FIG. 8A) in a patient vasculature. In another exemplary approach, the balloon (702) may be inflated with carbon dioxide or another relatively biologically inert gas.

The imaging element (708) may comprise a distal end of an optical fiber, in which case an image transmission line (not shown in FIG. 7) facilitating communication between the imaging element (708) and a proximal end of the catheter comprises an optical fiber. In another example, a charge-coupled-device (CCD) or infrared imaging chip may be employed as the imaging element (708), in which case an image transmission line may comprise an electronic data transmission wire. A lighting element (710) may also be paired with the imaging element to provide illumination or radiation appropriate for capturing images in the given tissue cavity. In the case of an optical fiber distal end as an imaging element (708), the lighting element (710) may comprise an emitter of light, such as a small light bulb, light emitting diode, or end of another optical fiber distal end in communication with an emitter or light. A light energy transmission line (not shown in FIG. 7) may comprise an optical fiber, electronic lead wire, or the like to transmit the appropriate lighting energy to the lighting element (710) from the proximal end of the catheter. In another example, the lighting element (710) comprises an emitter of infrared-spectrum radiation and the imaging element (708) comprises an infrared-detecting imaging element to enable infrared-spectrum visualization within the geometrically prescribed field of view. Suitable infrared emitters and detectors are well known in the art and available from suppliers such as CardioOptics of Boulder, Colo.

The imaging element (708) may comprise a lens, filter, mirror, or other structure configured to control the field of view or focal length of the associated imaging element (708). Further, a lens, filter, mirror, or other structure may be positioned distally from the imaging element (708) for similar purposes. The utilization of an imaging element (708) located at the distal end of a medical instrument, such as a balloon catheter, for purposes of visualizing objects from the point of interest is referred to herein as “direct visualization.” In other words, “direct visualization” is used in reference to placing an imaging “eye” distally to the location of tissue treatment interest.

In one exemplary illustration, the elongate tubular member (718) comprises a conventional polymeric material, such as that sold under the trade name “Pebax™” by Atofina Corporation, which is suitable for use inside of animals and has desirable mechanical and manufacturing properties. In the case of optical fiber, glass fibers, such as those conventionally utilized in endoscopes, may be utilized, or more flexible polymeric optical fibers, such as those available from Nanoptics Corporation of Gainesville, Fla., may be utilized.

The balloon (702) has an exterior surface defining a channel (750) extending axially between a proximal end (704) and a distal end (706) of the balloon (702). The channel (750) may thereby permit fluid communication between the distal end (706) and proximal end (704) when the balloon is inflated, e.g., within a vessel. For example, as shown in the section view of FIG. 8A, the balloon (702) cooperates with an interior surface (802) of a vessel (800) to define a plurality of axially extending channels (750). While the vessel (800) is illustrated as a tubular vessel, other exemplary vessels may define different sectional shapes or configurations. In this manner, the balloon (702) may permit fluid flow through the vessel (800) while it is inflated and engaged with the interior surface (802) of the vessel (800).

The balloon (702) may thus be employed to position the elongate member (718) and/or balloon catheter (720) at a desired position within the vessel (800), without blocking flow through the vessel (800). For example, the balloon (702) may generally prevent contact between the distal portion (700) of the catheter and/or a tip of the elongate member (718) and/or balloon catheter (720) with the interior surface (802) of the vessel (800). The positioning of the elongate member (718) with the balloon (702) may be useful to avoid damaging the vessel (800) through contact with the interior surface (802) thereof, e.g., while inserting the elongate member (718) through the vessel (800). In other words, the balloon (702), which provides a relatively soft interface with the interior surface (802) of the vessel (800), may generally slide along the interior surface (802) as the elongate member (718) is inserted through the vessel (800). In one exemplary illustration, a hydrophilic coating is provided about the exterior surface of the balloon (702) to facilitate sliding the expanded balloon (702) along the interior surface (802). Moreover, the balloon (702) may generally facilitate a view of the surrounding environment of the inflated balloon (702), e.g., to permit viewing of the interior surface (802) of the vessel (800) or other features of a patient anatomy.

The balloon (702) may also be configured to limit internal pressure, e.g., to avoid balloon (702) rupture or overinflation. For example, a check valve may be provided, e.g., at the distal end (706) of the balloon, with a threshold setting configured to limit maximum pressure and alleviate risk of rupture.

As best seen in FIGS. 7 and 8A, upon inflation within the vessel (800), outer arc portions (730) of the balloon (702) may cooperate with inner arc portions (732) to define the channels (750). For example, as shown in the radial section of the balloon (702) in FIG. 8, the exterior surface of the balloon (702) includes a first arc portion (730) disposed radially outwardly in engagement with the interior surface (802) of the vessel (800). The exterior surface of the balloon (702) also includes a second arc portion (732) positioned radially inwardly from the first arc portion (730). The first arc portion (730) has a first radius R₁, which is greater than a second radius R₂ defining the second arc portion (732). The different magnitudes of the first and second radii R1, R2 result in a transition along the exterior surface of the balloon (702), which defines the channels (750). While four separate channels (750) are illustrated in FIGS. 7, 8A, and 8B, any number of channels may be employed that is convenient.

The structure of the balloon (702) may be any that is convenient to facilitate formation of the channels (750) upon inflation of the balloon (702) within a vessel (800). For example the balloon (702) may have an axially extending rib (734) positioned adjacent each channel (750), as best seen in FIG. 8A. The ribs (734) may generally comprise a relatively thicker material in relation to the adjacent portions of the balloon (702), which generally prevents full expansion of the balloon (702) adjacent the ribs (734) and preventing the areas of the balloon (702) adjacent the ribs from contacting the interior surface (802) of the vessel. Accordingly, the ribs (734) may each define a radially inner portion of their respective channel (750). Alternatively or in addition, a clip (760) (shown optionally in FIG. 8A) may generally pinch a small portion of the balloon (702) which is folded over upon itself, thereby urging the pinched portion of the balloon (702) away from the interior surface (802) of the vessel. In yet another example, radially extending ribs (not shown) may define a maximum diameter of the balloon (702) such that expansion of the balloon (702) adjacent the radially extending ribs is prevented.

In another exemplary illustration, portions of the balloon (702) may be bonded together to form the channels (750). For example, referring to FIG. 8A, an exterior surface of a first inner arc portion (732a) may be bonded to the exterior surface of an adjacent inner arc portion (732b), thereby generally “pinching” the adjacent inner arc portions (732a), (732b) together and forming the channel (750) upon expansion.

In another example, balloon (702) may be comprised of “strips” extending axially along the balloon (702), where adjacent strips have a different compliance or stretching characteristic, which results in corresponding differences in expansion of the relevant portions of the balloon (702). For example, strips extending axially along the inner arc portions (732) may be formed of a less compliant material than strips extending axially along the outer arc portions (730). More specifically, referring to FIG. 8A, radial arc portions (730) may be formed of a material having a lesser compliance than a material forming the radial arc portions (732). Accordingly, the balloon (702) is more easily stretched along the outer arc portions (730) than the inner arc portions (732), and upon inflation the outer arc portions (730) expand more rapidly, coming into contact with the interior surface (802) while the inner arc portions (732) remain out of contact with the interior surface (802).

As noted above, exemplary balloons, e.g., balloon (702), may generally define a single inflatable chamber in communication with at least one balloon sizing lumen (not shown in FIG. 8A). The single chamber of the balloon (702), for example, generally extends about an entire radial perimeter of the balloon (702). Accordingly, in such examples the entire balloon may generally inflate or deflate in a generally uniform fashion.

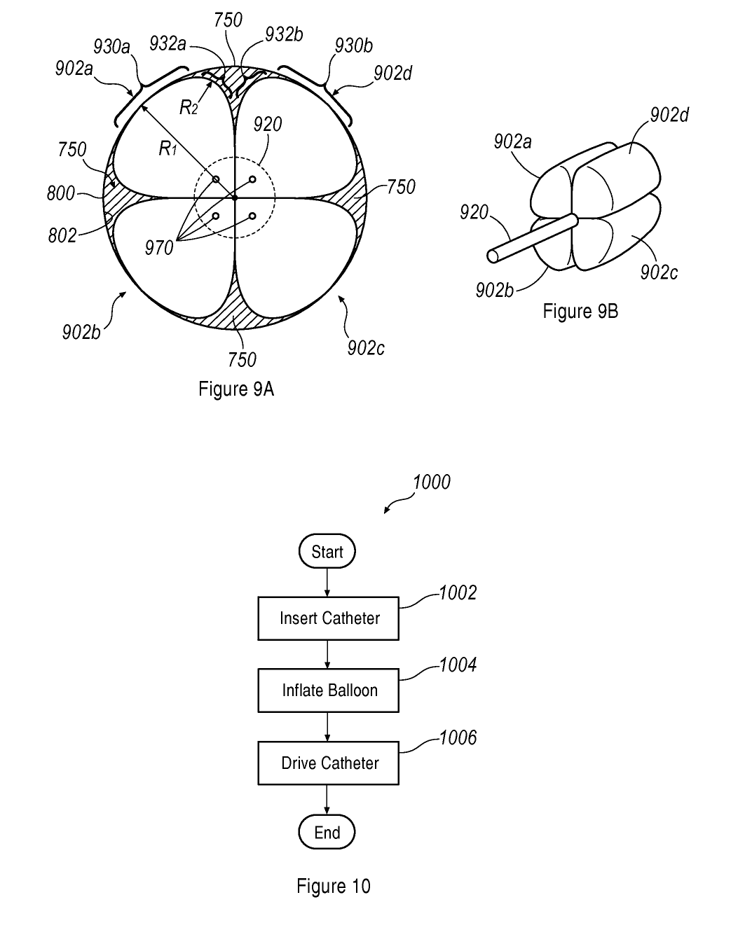

Turning now to FIGS. 9A and 9B, in another exemplary approach a balloon catheter may comprise a plurality of balloons (902a), (902b), (902c), (902d) (collectively, 902) that are separately expandable. The separate balloons (902) each collectively define channels (750) in the same fashion as described above regarding balloon (702). In contrast to the generally single inflatable chamber of the balloon (702), an exemplary balloon (902) comprises a plurality of separately expandable chambers (902a), (902b), (902c), (902d). Moreover, a balloon catheter (920) connected to or otherwise in communication with the balloon (902) may include a plurality of balloon sizing lumens (970), with each configured to supply a fluid to one of the chambers (902a), (902b), (902c), and (902d). In this manner, an inflation and/or internal pressure of each of the chambers (902a), (902b), (902c), and (902d) may be varied with respect to each other.

Similar to the exemplary approaches illustrated in FIGS. 7, 8A, and 8B, upon inflation within the vessel (800), the balloons (902a), (902b), (092c), and (902d) may cooperate to define channels (750) as a result of adjacent arc portions having different radii. For example, as best seen in FIG. 9A, outer arc portions (930) of the balloon (902) may cooperate with adjacent inner arc portions (932) to define the channels (750). More specifically, the exterior surface of the balloon (902a) includes a first arc portion (930a) disposed radially outwardly in engagement with the interior surface (802) of the vessel (800). The exterior surface of the balloon (902a) also includes a second arc portion (932a) positioned radially inwardly from the first arc portion (930a). The first arc portion (930a) has a first radius R₁, which is greater than a second radius R₂ defining the second arc portion (932a). The different magnitudes of the first and second radii R1, R2 result in a transition along the exterior surfaces of the adjacent balloons (902a) and (902d), thereby defining the channel (750) between the balloons (902a) and (902d). Moreover, the balloons (902b) and (902c) similarly cooperate with the adjacent balloons (902) to define the channels (750) there between.

While four separate channels (750) are defined by the four separate balloons (902a), (902b), (902c), and (902d) illustrated in FIGS. 9A and 9B, any number of channels may be employed that is convenient. Moreover, while the balloons (902) are shown each having similar shapes and sizes within the vessel (800), in other examples one or more of the balloons (902) may have a different size or shape relative to another one of the balloons (902).

Turning now to FIG. 10, an exemplary process (1000) of traversing a patient anatomy, e.g., vessel (800) of a patient's vasculature, is described. Process (1000) may begin at block (1002), where a catheter is inserted, e.g., into a vessel. For example, as described above a balloon catheter comprising a balloon (702) or (902) defining at least one channel (750) extending axially along the exterior surface between the distal end (706) and the proximal end (704) of the balloon. The balloon may define a generally single inflatable chamber, or may include a plurality of separately expandable chambers (902a), (902b), (902c), (902d). In examples where more than one expandable chamber is provided, an elongate member connected with the balloon may include a plurality of balloon sizing lumens, e.g., lumens (970), which are configured to supply a fluid to one or more of the chambers (902a), (902b), (902c), (902d). Accordingly, the internal pressure of the various chambers (902a), (902b), (902c), (902d) may be independently controlled.

Proceeding to block (1004), the balloon may be inflated. For example, as described above a balloon (702) or (902) may be inflated to engage an interior surface or wall (802) of a vessel (800) with the exterior surface of the balloon (702), (902). While the balloon (702), (902) is inflated and engaged with the interior surface (802) of the vessel, the channel(s) (750) permit fluid communication between the distal and proximal ends of the balloon (702), (902). In one example, as described above, adjacent radial sections of a balloon, e.g., arc portions (730), (930a), may have a different radius than an adjacent arc portion (732), (932a), respectively, thereby defining the channels (750). Accordingly, the balloon (702), (902) may be inflated to permit viewing of the interior of the vessel (800) or other portions of the patient's anatomy, or to permit driving the catheter along a centerline of the vessel (800) away from the interior surface (802) of the vessel (800), without blocking flow, e.g., of blood, through the vessel (800). In examples, where multiple chambers of a balloon are provided, e.g., as with balloon (902), the various chambers (902a), (902b), (902c), (902d) may be selectively inflated to different pressures to facilitate positioning of an associated elongate member, e.g., catheter (920). Process (1000) may then proceed to block (1006).

At block 1006, the catheter may be driven along the vessel. For example, as described above, exemplary balloon catheters may be inserted through vessel (800) while the balloon (702), (902) is inflated and the exterior surface of the balloon (702), (902) is engaged with the interior surface (802) of the vessel (800). In this manner, a distal portion (700) or a tip of the catheter may be generally spaced away from the interior surface (802) of the vessel (800) during insertion, thereby preventing contact with the interior surface (802) or wall of the vessel 800.

Exemplary balloons may be selectively inflated or deflated to facilitate navigation within a patient, e.g., as a catheter is being driven as described at block 1006 above. For example, internal pressure of a balloon may be increased or decreased to increase or decrease a size of the balloon, respectively, in order to allow movement of the balloon within a patient vasculature. More specifically, a patient vasculature may include vessels of different sizes or otherwise requiring altering an internal pressure of the balloon in order to allow passage of the balloon through the vasculature. Accordingly, exemplary balloons (702), (902) may be inflated or deflated while the balloon is being driven along a vessel. For example, in the exemplary approach illustrated in FIG. 9A, each of the balloons 902a, 902b, 902c, 902d have a balloon sizing lumen (970), and each of the balloon sizing lumens (970) allow fluid exchange with an external fluid supply (not shown). In one exemplary approach, one or more of the balloons (902) are deflated by allowing fluid to escape from the balloon(s) (902), thereby reducing a size of the balloon(s) (902) and permitting the balloon catheter to be navigated into a smaller vessel or otherwise within a patient vasculature. In one exemplary approach, all of the balloons (902a, 902b, 902c, 902d) are deflated, while in another example at least one balloon is deflated while the others are not. Accordingly, navigation of the balloon catheter may be aided by selectively inflating/deflating the balloons (902) individually.

Although exemplary illustrations have been described herein with reference to specific examples, many modifications therein will readily occur to those of ordinary skill in the art without departing from the inventive concepts taught herein. Accordingly, all such variations and modifications are included within the intended scope of the invention as defined by the following claims.

1. A system for traversing a vasculature, comprising:
an elongate tubular member having a distal end and defining a working lumen;
an inflatable balloon coupled to the distal end of the elongate tubular member, the balloon having a distal end and a proximal end;
wherein an exterior surface of the balloon defines at least one channel extending axially along the exterior surface between the distal end and the proximal end of the balloon, the channel configured to permit fluid communication between the distal and proximal ends of the balloon when the balloon is inflated within a vessel; and
wherein a radial section of the exterior surface of the balloon includes first and second arc portions having a first radius and a second radius, respectively, the first radius being a different magnitude than the second radius.

2. The system of claim 1, wherein the first and second arc portions are formed of first and second materials, respectively, the first material defining a different compliance than the second material.

3. The system of claim 1, wherein the balloon includes an axially extending rib adjacent the at least one channel, the rib defining a radially inner portion of the channel.

4. The system of claim 1, wherein the balloon includes a clip disposed about a folded portion of the balloon defining a radially inner portion of the channel.

5. The system of claim 1, wherein the elongate tubular member further defines at least one balloon sizing lumen configured to permit fluid exchange between the balloon and a fluid supply to facilitate inflation and deflation of the balloon.

6. The system of claim 5, wherein the balloon comprises a plurality of separately expandable chambers, and the elongate tubular member comprises a plurality of balloon sizing lumens each configured to permit fluid exchange between the balloon and a fluid supply.

7. The system of claim 1, wherein the balloon comprises a plurality of independently expandable chambers.

8. The system of claim 1, wherein the balloon comprises a single expandable chamber extending about an entire radial perimeter of the balloon.

9. The system of claim 1, wherein the balloon comprises a hydrophilic coating on the exterior surface of the balloon.

10. The system of claim 1, further comprising an imaging element disposed in an interior of the balloon.

11. The system of claim 10, wherein the imaging element comprises one of a charge-coupled device and an optical fiber.

12. The system of claim 1, further comprising a lighting element disposed in the interior of the balloon.

13. The system of claim 12, wherein the lighting element comprises one of an incandescent light source, a light-emitting diode, and an optical fiber.

14. The system of claim 1, wherein the elongate tubular member further defines at least one balloon sizing lumen configured to collect a fluid from the balloon to deflate the balloon.

15. The system of claim 14, wherein the balloon comprises a plurality of separately expandable chambers, and the elongate tubular member comprises a plurality of balloon sizing lumens each configured to collect a fluid from at least one of the plurality of separately expandable chambers to at least partially deflate at least one of the plurality of separately expandable chambers.

16. A catheter system, comprising:
an elongate tubular member having a distal end and defining a working lumen;
an inflatable balloon coupled to the distal end of the elongate tubular member, the balloon having a distal end and a proximal end, wherein an exterior surface of the balloon defines at least one channel extending axially along the exterior surface between the distal end and the proximal end of the balloon, the channel configured to permit fluid communication between the distal and proximal ends of the balloon when the balloon is inflated within a vessel, wherein a radial section of the exterior surface includes first and second arc portions having a first radius and a second radius, respectively, the first radius being a different magnitude than the second radius;
an imaging element disposed in an interior of the balloon; and
a tubular element defining a lumen between the distal end of the balloon and a distal end of the working lumen of the elongate tubular member.

17. The catheter system of claim 16, wherein the elongate tubular member further defines at least one balloon sizing lumen configured to supply a fluid to expand the balloon.

18. The catheter system of claim 17, wherein the balloon comprises a plurality of separately expandable chambers, and the elongate tubular member comprises a plurality of balloon sizing lumens each configured to supply a fluid to a corresponding one of the chambers.

19. A method for traversing a vessel in a body, comprising:
inserting a balloon catheter into the vessel, the balloon catheter comprising a balloon defining at least one channel extending axially along the exterior surface between the distal end and the proximal end; and
inflating the balloon to engage an interior wall of the vessel with the exterior surface of the balloon, wherein the channel permits fluid communication between the distal and proximal ends when the balloon is inflated within the vessel; wherein a radial section of the exterior surface after inflation includes first and second arc portions having a first radius and a second radius, respectively, the first radius being a different magnitude than the second radius.

20. The method of claim 19, further comprising establishing the balloon as comprising a plurality of separately expandable chambers, the elongate tubular member comprising a plurality of balloon sizing lumens each configured to supply a fluid to a corresponding one of the chambers.

21. The method of claim 20, further comprising one of selectively inflating and selectively deflating at least one of the plurality of separately expandable chambers to a different pressure than at least one other of the plurality of separately expandable chambers.

22. The method of claim 19, further comprising driving the balloon catheter along the vessel while the balloon is engaged with the interior wall of the vessel, thereby preventing a distal portion of the catheter from contacting the interior wall of the vessel.
